(19) United States
(12) Patent Application Publication
(10) Pub. No.: US 2025/0040792 A1
(43) Pub. Date: Feb. 6, 2025

(54) DISPOSABLE ENDOSCOPE SHEATH

(71) Applicant: Nizam Meah, Manvel, TX (US)

(72) Inventors: Sofia AGUILERA, Houston, TX (US); Travis DOWD, Austin, TX (US); Ariadna GOMEZ, Doral, FL (US); Emily McAMIS, Madison, WI (US); Nizam MEAH, Manvel, TX (US)

(21) Appl. No.: 18/790,938

(22) Filed: Jul. 31, 2024

Related U.S. Application Data
(60) Provisional application No. 63/516,567, filed on Jul. 31, 2023.

(51) Int. Cl. A61B 1/00

(52) U.S. Cl. CPC A61B 1/00135; A61B 1/00103

(57) ABSTRACT

A disposable endoscope sheath removably sheathes an endoscope used for an endoscopic procedure. The endoscope sheath includes a sheath body having a proximal end portion that connects to an endoscope controller device for controlling an endoscope sheathed within the endoscope sheath, and a distal end portion that is inserted within a subject. The sheath body comprises an elongate central portion and first and second wrapping flaps extending from opposite sides of the elongate central portion to respective free longitudinal edge margins. The sheath body is configured to releasably sheath the endoscope by wrapping the first and second wrapping flaps around the endoscope and securing the free longitudinal edge margins at a seam. The sheath body also includes channels for administering treatment to the subject. A distal endcap attaches to the distal end portion of the sheath body, and operably interfaces with the channels for performing the procedure.

### CROSS-REFERENCE TO RELATED APPLICATIONS

This application claims the benefit of U.S. Provisional patent application Ser. No. 63/516,567 filed on Jul. 31, 2023, the entire contents of which are hereby incorporated by reference.

### FIELD

The present disclosure relates to a disposable endoscope sheath for sheathing an endoscope.

### BACKGROUND

Endoscopes are commonly used for diagnosing and treating various medical conditions. There are many different endoscope types. For example, a colonoscope is a type of endoscope that can be used to diagnose conditions of a colon.

Endoscopes are generally tubular and can include long narrow channels used to facilitate diagnosis and treatment. These channels are difficult to access, resulting in endoscope disinfection processes that are time and resource intensive. For example, disinfecting a reusable colonoscope after every procedure wastes approximately 10-15 gallons of water per colonoscope. The disinfection process can take between 45 minutes to an hour. This disinfection process has over 100 different steps that must be followed meticulously by hospital technicians, or else bacteria and viruses can remain within the channels of the colonoscopes. One technique known to reduce the resources required for disinfecting an endoscope after use, is to sheath the endoscope used during a procedure.

### SUMMARY

Aspects of the present disclosure provide a disposable endoscope sheath for removably sheathing an endoscope during an endoscopic procedure.

In one aspect, a disposable endoscope sheath for removably sheathing an endoscope comprises a sheath body configured to define an endoscope lumen and to sheath the endoscope in the endoscope lumen. The sheath body comprises a proximal end portion and a distal end portion spaced apart along a longitudinal axis. The proximal end portion is configured to connect to an endoscope controller device, and the distal end portion is configured to be inserted into a subject. The sheath body comprises an elongate central portion and first and second wrapping flaps extending from opposite sides of the elongate central portion to respective free longitudinal edge margins. The sheath body is configured to releasably sheath the endoscope by wrapping the first and second wrapping flaps around the endoscope and securing the free longitudinal edge margins of the first and second wrapping flaps at a seam.

In another aspect, a disposable endoscope sheath mold for forming a disposable endoscope sheath comprises a sheath body including an elongate central portion and first and second wrapping flaps on opposite sides of the elongate central portion. The disposable endoscope sheath mold comprises a mold cavity configured to form an outer surface of the elongate central portion and the first and second wrapping flaps. The mold cavity comprises a first end portion and a second end portion spaced apart along a longitudinal axis. A mold core is configured to be impressed upon the mold cavity to form an inner surface of the elongate central portion and the first and second wrapping flaps. When the mold core is impressed upon the mold cavity, the mold core and mold cavity define a material receiving space between the mold cavity and the mold core. The material receiving space defines a thickness of the sheath body.

In another aspect, a method for using an endoscope comprises loading the endoscope radially onto a central portion of a disposable endoscope sheath. First and second wrapping flaps of the disposable endoscope sheath are wrapped around the endoscope. The first and second wrapping flaps are joined together at a longitudinal seam.

Other objects and features will be in part apparent and in part pointed out hereinafter.

### BRIEF DESCRIPTION OF THE DRAWINGS

FIG. 1 is an elevation of a disposable endoscope sheath including a sheath body and a distal endcap, as well as a proximal endoscope controller device coupled to a proximal end portion of the disposable endoscope sheath;

FIG. 2 is a perspective of the sheath body in an open-configuration with an endoscope loaded into an open endoscope lumen thereof;

FIG. 3 is a perspective of the sheath body similar to FIG. 2, but wherein the sheath body is closed around the endoscope;

FIG. 4 is an elevation of the disposable endoscope sheath in the open-configuration;

FIG. 5 is an elevation of the disposable endoscope sheath in a closed-configuration;

FIG. 6 is an end view of the distal endcap;

FIG. 7 is a cross-section taken in the plane of line 7-7 of FIG. 6;

FIG. 8 is a cross-section taken in the plane of line 8-8 of FIG. 6;

FIG. 9 is an exploded elevation of a mold core and a mold cavity of a disposable endoscope sheath mold; and

FIG. 10 is an elevation of a removable end block of the disposable endoscope sheath mold;

Corresponding reference characters indicate corresponding parts throughout the drawings.

### DETAILED DESCRIPTION

This disclosure generally pertains to a disposable endoscope sheath that can be used to sheath an endoscope during an endoscopic procedure. As with prior endoscope sheaths, the disposable endoscope sheath device of the present disclosure forms a sealed sheath around the endoscope so that during the procedure the endoscope itself is not exposed to contaminants. The sheath is made of low-cost, disposable materials so that it can be discarded after use, rather than sterilized through resource-intensive sterilization techniques. As will be explained in further detail below, the disposable endoscope sheath of the present disclosure allows for many of the typical endoscope functions to be performed through channels in the sheath, thereby providing benefits in terms of case of cleaning, without reducing the functional capabilities of the endoscope.

The inventors have recognized that one difficulty with current endoscope sheaths is installation. With current endoscope sheath designs, the endoscope must be inserted longitudinally into the close-fitting endoscope lumen. However, once a certain length of endoscope (e.g., 10 cm) is inserted into the close-fitting endoscope lumen, frictional resistance to further insertion becomes very great. It is difficult to advance the endoscope further into the lumen, and in some instances, the forces required to overcome the frictional resistance will cause damage to the endoscope.

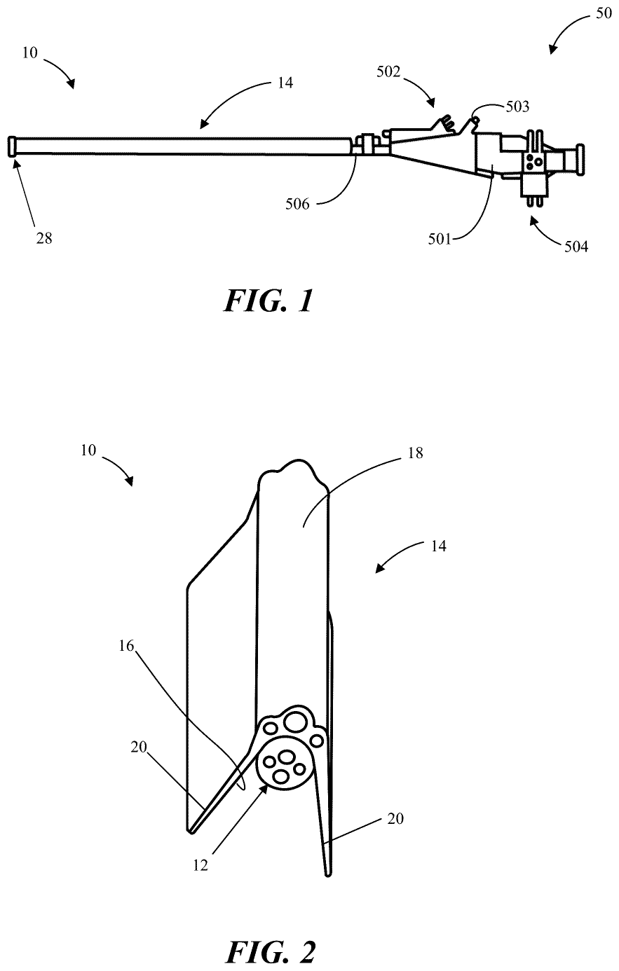

Referring to FIGS. 1-3, an exemplary embodiment of a disposable endoscope sheath in accordance with the present disclosure is generally indicated at reference number 10. Instead of using a tube-shaped sheath body with a permanently closed endoscope lumen to encase an endoscope, the endoscope sheath 10 of the present disclosure employs an open sheath body 14. The open sheath body 14 allows an endoscope 12 to be loaded radially into an open endoscope lumen 16 of the disposable endoscope sheath 10 (instead of being inserted longitudinally into a close-fitting lumen). As explained more fully below, the sheath body 14 is subsequently configured to be wrapped around the endoscope 12 and sealed along a longitudinal seam, whereby the sheath body forms a closed, sealed endoscope lumen 16 in which the endoscope is sheathed in a close-tolerance fit. The disposable endoscope sheath 10 further comprises a disposable distal end cap 28 configured to be installed on the distal end portion of the sheath body 14 for sealing the distal end of the endoscope lumen 16. The sheath body 14 and distal end cap 28 are configured to collectively define working channels that act as a disposable substitute for the normal working channels of the endoscope 12. The proximal end portion of the disposable endoscope sheath 10 is configured to connect to an endoscope controller device 50, which provides interfaces to the working channels, among other things.

Referring to FIG. 2, the sheath body 14 comprises an elongate central portion 18 and first and second elongate wrapping flaps 20 extending outward from the sides of the elongate central portion 18. The sheath body 14 has length extending along a longitudinal axis from a distal end portion to a proximal end portion. In one or more embodiments, the cross-sectional shape of the sheath body 14 is substantially uniform along the length. The length of the sheath body 14 corresponds to a length of the endoscope 12 configured to be inserted into the subject, so that when the endoscope 12 is sheathed within the endoscope lumen 16, the elongate central portion 18 covers at least the entire length of the endoscope 12 that may enter a subject's body. In the open-configuration, the sheath body 14 is essentially U-shaped in cross-section, with an opening between the wrapping flaps 20 through which the endoscope 12 is passable radially into the endoscope lumen 16. In the closed-configuration, the sheath body 14 has a generally annular shape in cross-section and fully circumscribes the endoscope lumen 16 for encasing the endoscope 12 therein. In one or more embodiments, a radial wall thickness of the closed sheath body 14 is non-uniform around its circumferences. The non-uniform wall thickness allows for the inclusion of working channels 22, 24, 26 and facilitates a sealed longitudinal seam that connects the wrapping flaps 20, as will be described in further detail below.

The central portion 18 of the sheath body 14 has a generally U-shaped cross-sectional shape with flexible opposing arms that connect the central portion 18 to the wrapping flaps 20. In cross-section, the elongate central portion 18 has a wall thickness that is greatest near the middle and tapers outward along the flexible opposing arms. Between the tapered arms, the central portion 18 defines a plurality of working channels 22, 24 and 26 for administering treatment to the subject. In one or more embodiments, each of the working channels 22, 24, 26 is formed from a tube over-molded with silicone to from the sheath body 14. Suitably, each of the channels 22, 24, 26 extends from a first port at the proximal end portion of the sheath body 14 to a second port through the distal end portion of the sheath body. The proximal ports of the channels 22, 24, 26 are configured for fluid connection to the endoscope controller device 50, and the distal ports are configured for connection to the distal endcap 28, as will be described in further detail below. During an endoscopic procedure, the working channels 22, 24, 26 are configured to pass fluids and/or instrumentations between the endoscope controller device 50 and a cavity inside a patient.

In an exemplary embodiment, the central portion 18 of the sheath body 14 comprises a low-pressure water and air channel 22, a suction/biopsy channel 24, and a high-pressure water channel 26. The suction/biopsy channel 24 has a greater inner diameter than the low-pressure water and air channel 22 and the high-pressure water channel 26. The suction/biopsy channel 24 is dual-purpose. Via the endoscope controller device 50, an operator is configured to draw suction through the suction/biopsy channel 24 to remove fluid and tissue from the subject. In addition, the suction/biopsy channel 24 can be used as the lumen through which a biopsy instrument is inserted into the subject. The low-pressure water and air channel 22 is configured to carry either low-pressure water or air from the endoscope controller device 50 to the subject. The high-pressure water channel 26 is configured to carry high-pressure water from the endoscope controller device 50 to the subject. Those skilled in the art will recognize that air, water and suction/biopsy channels are typically present inside an endoscope. However, the inventors believe that the process for cleaning these channels after each use is too time and resource-intensive. By including the working channels 22, 24, 26 in the disposable endoscope sheath body 14, the same low-pressure air, low-pressure water, suction, biopsy and high-pressure water functionality may be provided without the cleaning requirements. Instead, the sheath body 14 which defines the channels 22, 24 and 26, can simply be disposed of after one use (i.e., the disposable endoscope sheath 10 can be a single use medical device).

In an exemplary embodiment, the endoscope controller device 50 comprises an ergonomic handle 501, an accessory biopsy port 502, an endoscope biopsy port 503, a valve system 504, and a pneumatic connector 506. Broadly, the endoscope controller device 50 is configured to allow for physician operation of the channels 22, 24, and 26, and configured for facilitating pneumatic and biopsy capabilities through the sheath 10.

The handle 501 is configured to interface with (e.g., by snap-fit) a control section of the endoscope 12. The handle 501 is configured such that a physician can easily and comfortably place their hand on the endoscope control section. In an embodiment, the handle comprises the valve system 504. The valve system 504 in a particular embodiment comprises two valves. A first valve allows for suction to be applied through the sheath 10 when actuated by a user pressing on the valve, and a second valve allows for air insufflation through the sheath when the user lightly depresses the valve and water insufflation when the user depresses the valve further. The accessory biopsy port 502 can also be clipped onto the endoscope 12 to allow for introduction of biopsy instruments into the sheath 10 through the same channel through which suction is produced. The accessory biopsy port 502 is configured to connect to the suction/biopsy channel 24 via tubing. In the illustrated embodiment, the accessory biopsy port has an angled neck through which a biopsy instrument can be inserted into the sheath's suction channel 24.

The pneumatic connector 506 comprises a barbed connector piece that connects to the sheath and tubing from the valves, so that different pneumatic functionalities actuated from the valves can be transferred to the channels in the sheath without air or water escape.

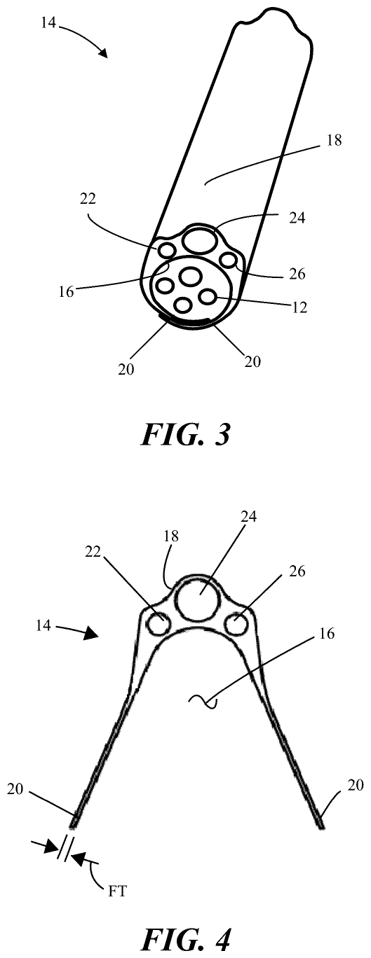

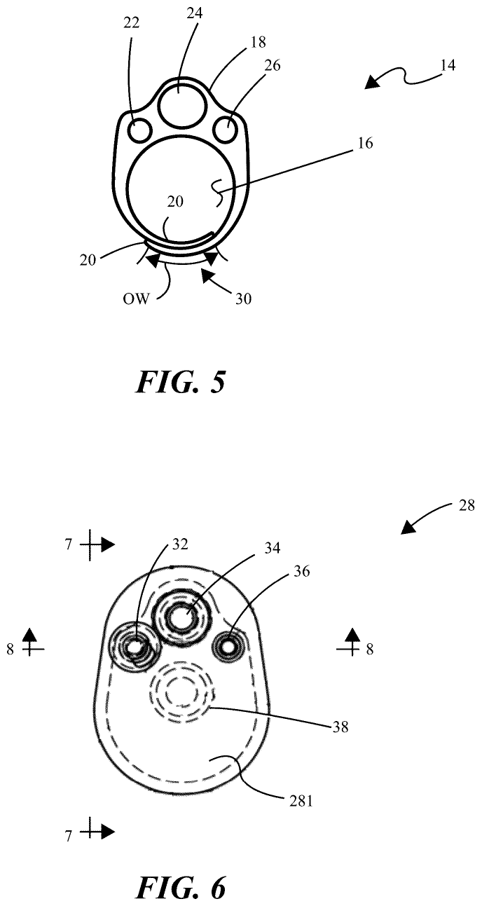

Referring to FIGS. 4 and 5, the tapered arms of the central portion 18 are connected to the wrapping flaps 20 such that the first and second wrapping flaps 20 extend from opposite sides of the elongate central portion 18 to respective free longitudinal edge margins. The wrapping flaps 20 are configured to be wrapped around a circumference of the endoscope 12 and secured together at a longitudinal seam 30 that extends along the entire length of the sheath body 14. In the illustrated embodiment, the seam 30 is an overlapping seam where the first and second wrapping flaps 20 are joined together at an overlap. However, it is contemplated that sheath bodies could be configured to form other types of seams after being wrapped around an endoscope without departing from the scope of the disclosure. FIGS. 2 and 4 show the first and second wrapping flaps 20 in an open-configuration, where the endoscope 12 can be loaded radially into the open endoscope lumen 16. FIGS. 3 and 5 show the first and second wrapping flaps 20 in a closed-configuration in which the sheath body 14 is configured to sheath the endoscope 12 inside a closed endoscope lumen 16. In cross-section, the first and second wrapping flaps 20 each have a wrapping flap thickness FT. In one or more embodiments, the wrapping flap thickness FT is an inclusive range from 0.5 mm to 1.25 mm.

When the wrapping flaps 20 are wrapped around the endoscope 12 and secured to one another at the seam 30, one flap is positioned radially inboard of the other. The inner flap 20 has an inner surface facing the endoscope, and the outer flap 20 has an inner surface facing the inner flap along the overlap seam 30. As shown in FIG. 5, the overlap seam 30 defines an overlap width OW. In one or more embodiments, the overlap width OW is in an inclusive range from 13 mm to 25 mm. Along the overlap width OW, the first and second wrapping flaps 20 are scalingly joined together by a pressure sensitive adhesive (PSA). In other words, at the scam 30, the flaps 20 are adhered together so as (i) not to move in relation to one another during an endoscopic medical procedure and (ii) to seal the interface between the two flaps 20 to prevent ingress of bodily fluids or other contaminants through the seam 30. In certain embodiments, the inner flap is also joined to the endoscope 12 with PSA.

It is envisioned that the sheath body 12 could be used with endoscopes of different sizes by varying the overlap width OW. For example, for an endoscope with a larger outer diameter, the wrapping flaps 12 could be joined together along an overlap scam 30 having a relatively small overlap width OW, whereas for an endoscope with a smaller outer diameter, the wrapping flaps 20 could be joined together along an overlap seam 30 having a relatively large overlap width OW. In one or more embodiments, the sheath body 14 is configured to fit endoscopes in an inclusive outer diameter size range from 9 mm to 13.5 mm. A person of ordinary skill in the art will understand that sheath body 14 may also be configured to fit endoscopes having outer diameters not in the 9 mm to 13.5 mm range without departing from the scope of the disclosure.

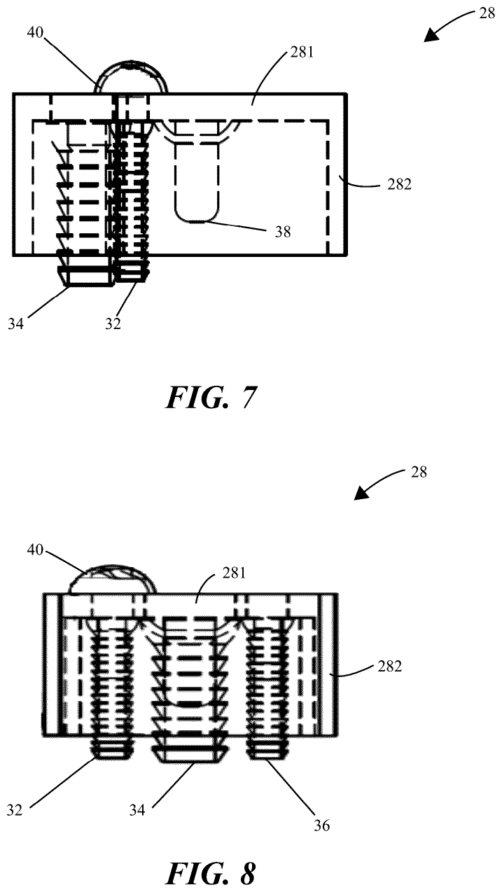

Referring to FIGS. 6-8, the distal endcap 28 is configured to releasably attach to the distal end portion of the sheath body 14. In the illustrated embodiment, the distal endcap 28 comprises an end wall 281 and a shroud 282 extending proximally from the end wall. The end wall 281 is configured to enclose the distal end of the endoscope lumen 16, and the shroud 282 is configured to mate with the distal end portion of the sheath body 14. In the illustrated embodiment, the distal endcap 28 further comprises a central alignment peg 38 extending proximally from the distal end wall at a central location inwardly spaced from the shroud 282. During use, the alignment peg 38 is configured to be received in the distal end portion of the endoscope lumen 16. In a preferred embodiment, the distal endcap 28 is manufactured out of a clear biocompatible material such as polymethyl methacrylate (PMMA). Hence, during use, the endoscope 12 can visualize images distal of the endcap 28 through the clear biocompatible material.

The distal endcap 28 is configured for sealing the distal end portion of the endoscope lumen 16. This prevents entry of bodily fluids into the endoscope lumen 16 where they could contaminate the endoscope 12 and its internal channels. In certain embodiments, the distal endcap 28 is configured to be joined and sealed to the distal end portion of the closed sheath body 14 using an adhesive or other bonding agent. Such adhesive may be applied to the exterior of the alignment peg 38, interior of the shroud 282, and/or exterior of the distal end portion of the closed sheath body 14. Further, the distal endcap 28 is configured to be friction-fit to the distal end portion of the closed sheath body 14, and a watertight seal of an interface between the sheath body and the distal endcap is made automatically by virtue of the friction-fit and adhesive. For example, the alignment peg 38 is configured to friction-fit inside the distal end portion of the endoscope lumen 16, and/or the distal end portion of the closed endoscope sheath 14 is configured to friction-fit inside the shroud 282. It is also contemplated that one or more compressible gaskets could be incorporated into the distal endcap 28 or distal end portion of the sheath body 14 for sealing the interface between the sheath body 14 and the distal endcap 28.

In the illustrated embodiment, the distal endcap 28 includes three barbed tubing connectors 32, 34, 36. The barbed connectors 32, 34, 36 are configured to interface with respective channels 22, 24, 26 of the sheath body 14. That is, each barbed connector 32, 34, 36 is configured to press-fit into the respective channel 22, 24, 26. Suitably, each barbed connector 32, 34, 36 is hollow and defines a connector lumen that opens through the end wall 281 of the distal endcap 28. During an endoscopic procedure, the barbed connectors 32, 34, 36 provide fluid communication between the working channels 22, 24, 26 and a cavity inside a patient.

The distal endcap 28 further comprises a water diverter 40 formed on the distal side of the end wall 281, extending across part of the outlet of the barbed connector 32 for the low-pressure water and air channel 22. The water diverter 40 is configured to divert water flowing longitudinally through the low-pressure water and air channel 22 and barbed connector 32 to flow in more radial direction across the distal face of the end wall 281. During use, the water diverter 40 directs from the low-pressure water and air channel 22 over the distal face to clean the distal face.

The disposable endoscope sheath 10, as discussed above, is configured to removably sheath various types of endoscopes during medical procedures. A method of using an endoscope for a procedure is further described below. For purposes of completeness, the method described below begins with a step of applying the PSA to the sheath body 14. In this case, the PSA may be applied by a manufacturer with release strips to cover the adhesive surfaces as explained in further detail below.

Initially, the manufacturer prepares the sheath body 14 for a medical practitioner to install on the endoscope 12 by applying a biocompatible PSA to one or both of the wrapping flaps 20. In an exemplary, embodiment, the manufacturer applies PSA to both the inner and outer surfaces of the inner wrapping flap 20 and the inner surface of the outer wrapping flap 20. In the currently preferred embodiment, the manufacturer applies adhesive in bands that extend lengthwise from the distal end portion to the proximal end portion of the sheath body 14, and which are approximately 12 millimeters wide. In one embodiment, the PSA may be left overnight to cure. Release liners may be added to an exposed top of the PSA layer to preserve adhesive strength.

When the PSA has properly cured, the sheath body 14 is installed on the endoscope 12 by a medical practitioner in the field, e.g., at the hospital or operating room facility, where the endoscope sheath 10 will be used. At this stage, the sheath body 14 is in the open-configuration. Any release liners covering the PSA may be removed to expose the PSA layer. The endoscope 12 is then loaded radially into the open endoscope lumen 16. When the endoscope 12 is properly supported against the inner surface of the elongate central portion 18, the inner wrapping flap 20 can be wrapped around the endoscope 12. The PSA on the inner surface of the inner wrapping flap 20 adheres to the endoscope 12 to fix the endoscope 12 and sheath body 14 in relation to one another. Subsequently, the outer wrapping flap 20 is wrapped around the endoscope 12 and pressed against the inner wrapping flap 20 to make the overlapping seam 30. The activated PSA joins the first and second wrapping flaps 20 at the seam 30 and seals the interface between the wrapping flaps 20.

Next a medical practitioner installs the distal endcap 28 onto the distal end portion of the sheath body 14. In one or more embodiments, the medical practitioner applies biocompatible sealant to one or more interface surfaces of the distal endcap 28 before press-fitting the distal endcap 28 onto the distal end portion of the sheath body 14. Press-fitting the distal endcap 28 onto the distal end portion of the sheath body 14 inserts the alignment peg 38 into the distal end portion of the endoscope lumen 16 and inserts the barbed connectors 32, 34 and 36 into the channels 22, 24 and 26. The sealant seals the interface between the distal endcap 28 and the sheath body 14.

As shown in FIG. 1, the medical practitioner also connects the proximal end portion of the sheath body 14 to the endoscope controller device 50 to facilitate the use of the endoscope 12. During the procedure, the endoscope controller device 50 can be connected to external devices such as suction devices, water containers, air pumps, etc. The endoscope controller device 50 may include a control handle, a valve system, a biopsy port and a sheath connector to facilitate pneumatic and biopsy capabilities through the disposable endoscope sheath 10. The control handle of the endoscope controller device 50 includes the valve system for controlling flow within the channels 22, 24 and 26. In one embodiment, the valve system includes two valves, one of which allows for suction to be applied through the suction/biopsy channel 24 when actuated by pressing on the valve, and another which allows for air insufflation through the low-pressure water and air channel 22 when the valve is lightly depressed and water insufflation through the low-pressure water and air channel 22 when the valve is depressed further. A water jet connection can be introduced to the high-pressure water channel 26. The biopsy port can be clipped onto the endoscope 12 to allow for the introduction of biopsy instruments into the disposable endoscope sheath 10 through the suction/biopsy channel 24. The valve system and biopsy port are connected to the disposable endoscope sheath 10 by a sheath connector. In the preferred embodiment, the sheath connector is a dual-channel connector with barbs on each side to facilitate connection between the endoscope controller device 50 and the suction/biopsy channel 24 and low-pressure water and air channel 22 of the disposable endoscope sheath 10 whilst maintaining fluid-tightness.

With the endoscope 12, sheathed within the disposable endoscope sheath 10 and connected to the distal endcap 28 and endoscope controller device 50, a procedure such as diagnosing the subject or administering treatment to the subject is conducted. With the distal end portion inserted to a subject, a physician may use the endoscope controller device 50 to control functionality of the channels 22, 24 and 26 that are used to perform the procedure. For example, a biopsy instrument can be inserted within the suction/biopsy channel 24 to perform a biopsy procedure on the subject. At the conclusion of the procedure, the distal end portion is removed from the subject, the distal endcap 28 is removed from the disposable endoscope sheath 10, and the proximal end portion is disconnected from the endoscope controller device 50. After the procedure, the endoscope 12 is removed from the disposable endoscope sheath 10 by separating the first and second wrapping flaps 20. Further, the disposable endoscope sheath 10 is disposed of.

A method of manufacturing the disposable endoscope sheath 10 is further described in detail below. The method involves using a disposable endoscope sheath mold 42 to form the disposable endoscope sheath 10 from a single piece of monolithic material such as biocompatible silicone. In general, the method and equipment for manufacturing described herein are configured for forming an open (U-shaped) endoscope sheath body 14.

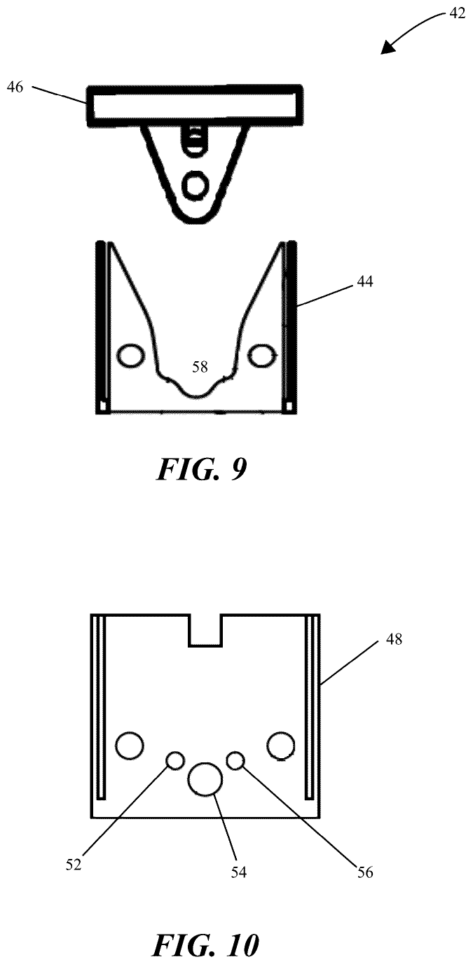

FIGS. 9 and 10 show the disposable endoscope sheath mold 42 for forming the disposable endoscope sheath 10. The disposable endoscope sheath mold 42 includes a mold cavity 44, a mold core 46, and first and second removable end blocks 48. Further, the disposable endoscope sheath mold 42 can include tubing retainers 52, 54 and 56 for holding tubing within the mold cavity 44, such that the tubing can be over-molded to form the channels 22, 24 and 26 in the sheath body 14.

The mold cavity 44 defines a mold surface configured to form an outer surface of the elongate central portion 18 and the first and second wrapping flaps 20. The first and second removable end blocks 48 are configured to releasably couple to first and second end portions of the mold cavity 44. The first and second removable end blocks 48 couple to the first and second end portions of the mold cavity 44 to close off the ends of the mold 42. The tubing retainers 52, 54, 56 are configured to be above the upward facing mold surface of the mold cavity 44 for holding tubing in the mold above the mold surface. During use of the mold, three lengths of tubing extend from one tubing retainer 52, 54, 56 in the first end block 48 to a corresponding tubing retainer 52, 54, 56 in the second end block 48. In a preferred embodiment, the first and second removable end blocks 48 include pegs that fit into receivers on the mold cavity 44 for retaining and aligning the end blocks 48 to the mold cavity 44.

The mold core 46 is configured to be impressed upon the mold cavity 44 to form an inner surface of the elongate central portion 18 and the first and second wrapping flaps 20. The mold core 46 has a length that protrudes from the first and second end portions of the mold cavity 44 to facilitate removal of the formed disposable endoscope sheath 10. Further, the mold core 46 can include notches to fit to the first and second removable end blocks 48 for standardizing alignment of the mold core 46.

When the mold core 46 is impressed upon the mold cavity 44, the mold core 46 and mold cavity 44 define a material receiving space 58 between the mold cavity 44 and the mold core 46. Further, the material receiving space 58 defines a thickness of the sheath body 14. The material receiving space 58 includes first and second tapered portions configured to define arms along which the thickness of the sheath body 14 tapers toward the first and second wrapping flaps 20. The tube retainers 52, 54, 56 are configured to align with the material receiving space 58 for holding the tubing in the material receiving space 58.

In an exemplary embodiment, the disposable endoscope sheath 10 is formed by coupling the first and second removable end blocks 48 to the first and second end portions of the mold cavity 44 and installing tubing in the tubing retainers 52, 54 and 56. The sheath body 14 is formed by pouring a biocompatible liquid silicone into the mold cavity 44 and impressing the mold core 46. The silicone is cured to form the disposable endoscope sheath 10. The mold core 46 and first and second removable end blocks 48 are separated from the disposable endoscope sheath mold 42 to remove the disposable endoscope sheath 10 from the disposable endoscope sheath mold 42.

Alternative methods for forming the disposable endoscope sheath 10 may be used. For example, instead of forming the disposable endoscope sheath 10 as one part, the wrapping flaps 20 may be manufactured separate from the sheath body 14 and further connected to form the disposable endoscope sheath 10. Moreover, the disposable endoscope sheath 10 may include a sheath body 14 with a uniform cross-section that enables the disposable endoscope sheath 10 to be formed by other large-scale manufacturing processes such as extrusion for accommodating different endoscope lengths.

Exemplary methods for manufacturing the distal endcap 28 will now be described. In one embodiment, the distal endcap 28 is manufactured via stercolithography (SLA) printing. During this process, a 3D CAD file for the distal endcap 28 is produced, aligned, printed, washed, and then cured. This method is particularly suitable for rapid prototyping production. In another embodiment, the distal endcap 28 is manufactured via injection molding. During the injection molding process, a mold for the distal endcap 28 is produced, and then material used for forming the endcap (e.g., PMMA) is injected into the mold after which it will then be cooled and demolded. It is also envisioned that, once the endcap 28 has been manufactured, a thin layer of silicone sheath will be applied to the endcap. The thin layer of silicone sheath will be maintained on the endcap 28 with a silicone release liner that maintains adhesive properties without fusing to the endcap.

Exemplary methods for forming the endoscope control device 50 will now be described. 3D CAD files of the ergonomic handle 501, accessory biopsy port 502, endoscope biopsy port 503, valve system 504, and pneumatic connector 506 are initially produced using a CAD software. The handle 501 in one embodiment is manufactured using FDM printing. The accessory biopsy port 502, endoscope biopsy port, valve system 504 and pneumatic connector 506 in one embodiment, are printed using SLA printing to obtain tight tolerances required for valve functioning and achieving pneumatic capabilities. It is also contemplated that in another method, the components of the endoscope control device 50 would be made using aluminum for achieving high fidelity, autoclavability, and reusability. In this embodiment, the handle 501 is formed using a custom injection mold, whereas the other components would be formed using aluminum machining techniques.

Having described the disclosure in detail, it will be apparent that modifications and variations are possible without departing from the scope of the disclosure defined in the appended claims.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved and other advantageous results attained.

As various changes could be made in the above products and methods without departing from the scope of the disclosure, it is intended that all matter contained in the above description and shown in the accompanying drawings shall be interpreted as illustrative and not in a limiting sense.

1. A disposable endoscope sheath for removably sheathing an endoscope, the disposable endoscope sheath comprising:
a sheath body configured to define an endoscope lumen and to sheath the endoscope in the endoscope lumen, the sheath body comprising a proximal end portion and a distal end portion spaced apart along a longitudinal axis, the proximal end portion configured to connect to an endoscope controller device and the distal end portion configured to be inserted into a subject, the sheath body comprising an elongate central portion and first and second wrapping flaps extending from opposite sides of the elongate central portion to respective free longitudinal edge margins, the sheath body configured to releasably sheath the endoscope by wrapping the first and second wrapping flaps around the endoscope and securing the free longitudinal edge margins of the first and second wrapping flaps at a seam.

2. The disposable endoscope sheath of claim 1, wherein the first and second wrapping flaps are configured to be wrapped around the endoscope such that the first and second wrapping flaps form an overlapping seam.

3. The disposable endoscope sheath of claim 2, wherein the first and second wrapping flaps are configured so that the overlapping seam has an overlap width in an inclusive range of 13 mm to 25 mm.

4. The disposable endoscope sheath of claim 1, wherein the first and second wrapping flaps are configured to be secured together by a biocompatible pressure sensitive adhesive.

5. The disposable endoscope sheath of claim 1, wherein the elongate central portion has a generally U-shaped cross-sectional shape, including opposing arms connected to the first and second wrapping flaps.

6. The disposable endoscope sheath of claim 5, wherein in cross-section, the elongate central portion has a thickness, the thickness tapering outwardly along the flexible opposing arms.

7. The disposable endoscope sheath of claim 1, wherein the sheath body comprises a plurality of channels configured for administering treatment to the subject.

8. The disposable endoscope sheath of claim 7, further comprising a distal endcap configured to releasably attach to the distal end portion.

9. The disposable endoscope sheath of claim 8, wherein the distal endcap comprises a plurality of barbed connectors configured to interface with the plurality of channels.

10. The disposable endoscope sheath of claim 8, wherein the distal endcap comprises an endoscope alignment peg configured to align the distal endcap to the endoscope by reception in the endoscope lumen.

11. The disposable endoscope sheath of claim 8, wherein the distal endcap comprises a water diverter configured to direct water over a distal face of the distal endcap.

12. The disposable endoscope sheath of claim 1, wherein the sheath body is configured to fit endoscopes in an inclusive outer diameter size range of 9 mm to 13.5 mm.

13. The disposable endoscope sheath of claim 1, wherein when the endoscope is sheathed by the disposable endoscope sheath, the sheath body comprises an overlapping seam and a channel region opposite the overlapping seam.

14. A disposable endoscope sheath mold for forming a disposable endoscope sheath comprising a sheath body including an elongate central portion and first and second wrapping flaps on opposite sides of the elongate central portion, wherein the disposable endoscope sheath mold comprises:
a mold cavity configured to form an outer surface of the elongate central portion and the first and second wrapping flaps, the mold cavity comprising a first end portion and a second end portion spaced apart along a longitudinal axis; and
a mold core configured to be impressed upon the mold cavity to form an inner surface of the elongate central portion and the first and second wrapping flaps, wherein when the mold core is impressed upon the mold cavity, the mold core and mold cavity define a material receiving space between the mold cavity and the mold core, the material receiving space defining a thickness of the sheath body.

15. The disposable endoscope sheath mold of claim 14, further comprising first and second removable end blocks configured to releasably couple to the first and second end portions of the mold cavity.

16. The disposable endoscope sheath mold of claim 14, further comprising at least one tubing retainer configured to hold tubing in the mold cavity such that the tubing can be over-molded to form one or more channels in the sheath body.

17. The disposable endoscope sheath mold of claim 14, wherein the material receiving space includes first and second tapered portions configured to define arms for connecting the elongate central portion to the first and second wrapping flaps.

18. The disposable endoscope sheath mold of claim 17, wherein the material receiving space defines a wrapping flap thickness in an inclusive range of 0.5 mm to 1.25 mm.

19. A method for using an endoscope, wherein the method comprises:
loading the endoscope radially onto a central portion of a disposable endoscope sheath;
wrapping first and second wrapping flaps of the disposable endoscope sheath around the endoscope;
joining the first and second wrapping flaps together at a longitudinal seam.

20. The method of claim 19, further comprising conducting a procedure using the endoscope sheathed by the disposable endoscope sheath, and after conducting the procedure, removing the endoscope from the disposable endoscope sheath by separating the first and second wrapping flaps.
